(19) United States
(12) Patent Application Publication
(10) Pub. No.: US 2014/0251917 A1
(43) Pub. Date: Sep. 11, 2014

(54) Method for the treatment of multiple sclerosis

(71) Applicant: Marv Enterprises,LLC, El Paso, TX (US)

(72) Inventor: Mitchell S. Felder, El Paso, TX (US)

(21) Appl. No.: 14/343,904

(22) PCT Filed: Sep. 19, 2012
(86) PCT No.: PCT/US2012/056015

§ 371 Date: Mar. 10, 2014

Related U.S. Application Data
(60) Provisional application No. 61/537,913, filed on Sep. 22, 2011.

(51) Int. Cl. A61M 1/36

(52) U.S. Cl. CPC A61M 1/3687; A61M 1/3681; A61M 1/3618
USPC 210/695; 210/749; 210/748.16

(57) ABSTRACT

The present invention relates to a treatment of multiple sclerosis, and includes the extracorporeal treatment of one or more body fluids, such as, for example blood, cerebral-spinal fluid, or lymphatic fluid. A treatment is applied to the extracorporeal body fluid where the treatment targets at least one target multiple sclerosis antigen in the body fluid. The treatment can include creating an antibody-antigen moiety and then removing antibody-antigen moiety from the body fluid before returning the body fluid to a patient.

The present invention claims priority to U.S. 61/537913 filed 22 Sep. 2012.

### FIELD OF THE INVENTION

The invention relates to a device and method for the treatment of multiple sclerosis.

### BACKGROUND OF THE INVENTION

In the United States multiple sclerosis (MS) is one of the leading causes of neurologic impairment. The disease affects more than 300,000 patients, id has its highest incidence in young adults. Initial symptoms of multiple sclerosis usually commence before the age of 55 years. There is a peak incidence between the ages of 20 and 40. Women are affected approximately twice as often as men. The disease is believed to have an autoimmune etiology. Multiple sclerosis is much more common in persons of western European lineage who live in temperate zones.

Certain molecular organic compounds are implicated as causing or allowing multiple sclerosis, which in turn allows for the progression of the disease, with increasing morbidity and mortality.

### SUMMARY OF THE INVENTION

In general terms, the present invention relates to the treatment of multiple sclerosis, hereinafter abbreviated as “MS”. Specifically, the invention pertains to a method for the extracorporeal treatment of one or more body fluids (blood, cerebral-spinal fluid (CSF), or lymphatic fluid) in two stages characterized by removing a body fluid from a living body diseased with a type of MS, passing the body fluid (blood, CSF, or lymphatic fluid) through a first stage; applying a treatment to at least one or more target MS antigen(s) in the body fluid, in order to expedite the removal of the targeted MS antigen(s).

More specifically, the treatment comprises creating an antibody-antigen moiety during passage thereof through said first stage; passing the treated body fluid through a second stage; removing antibody-antigen moiety from the body fluid during passage through the second stage, and returning the purified body fluid to the body.

The invention is further characterized by targeting an antigen in the body fluid, with an antibody to allow and facilitate removal thereof in the second stage. The targeted antigens would include one, or a combination of targeted MS Antigen(s) involved in the pathologic development of MS:

Integrin

Osteopontin

Interleukin-23, Interleukin-17. Interleukin-12, Interleukin-15

Intrathecal Immunoglobulins IgG/Oligoclonal Bands

Glutamate

Matrix metalloproteinases (MMPs)

Myelin bask protein (MBP)

Peptidyl arginine deiminase 2 (PAD 2)

Beta-Chemokines: monocyte chemoattractant protein-1 (MCP-1); macrophage inflammatory protein (MIP): RANTES Regulated on Activation Normal T Cell Expressed and Secreted (CCL5)

Myelin-associated oligodendrocytic basic protein (MOBP)

N-Acetyl-Aspartate

VLA-4: very late antigen-4

Cytokines: IL15 and LPS-cytokine

Adhesion Proteins: ALCAM (Activated Leukocyte Cell Adhesion Molecule); CD166 (cluster of differentiation 166); CXCL12 (chemokine ligand 12)

Endothelin-1

Kallikreins: KLK1, KLK6

Chromogranin A

Myelin Protein TPPP/p25

sFas: soluble form of the Fas molecule

MIF: macrophage migration inhibitory factor

TNF-alpha: tumor necrosis factor-alpha

CCL2: chemokine ligand 2

T helper cells Th1 and Th17

Activated T Cells and B Cells

LINGO-1: Leucine-rich repeat and Ig domain containing NOGO receptor interacting protein-1

sVCAM-1: soluble vascular adhesion molecule

A1AC: alpha-1 a inchymotrypsin

A2MG: alpha-1 macroglobulin

Fibulin 1

NMO-IgG/Aquaporin-4 Antibodies: specifically in the Neuromyelitis Optica (NMO) variant of MS

Specifically, the method is further characterized by removing body fluid (blood, CSF, or lymphatic fluid) from a person to produce the extracorporeal bodily fluid; imposing a treatment acting on one or more antigen(s) of targeted MS antigen(s) in the body fluid, filtering or otherwise removing the treatment from the body fluid, and returning the body fluid to the patient after removing substantially all of the treatment in the second stage.

The method of the present invention comprises treating at least one component of a patient's body fluid extracorporeally with a designer antibody containing, an albumin-moiety which will create an albumin-antibody-MS antigen moiety, allowing for the efficacious dialysis of the resultant albumin-antibody-MS antigen compound.

More specifically, the method is characterized by removing body fluid from a person to produce the extracorporeal bodily fluid; directing a first antibody against the targeted MS antigen in the first stage of extra-corporeal treatment in the body fluid; in the second stage directing a second antibody conjugated with albumin and/or a protein against the targeted MS antigen thereby forming an albumin-antibody-MS antigen compound; removing at least a substantial portion of the albumin-antibody-MS antigen compound from the body fluid by dialysis, other filtering, or other means; and returning the body fluid to the patient.

Also, the method is characterized by testing the blood and/or CSF or lymphatic fluid to determine the efficacy of treatment before returning the body fluid to the patient,

### BRIEF DESCRIPTION OF THE DRAWINGS

FIG. 1 is a partial cross sectional view of a cylinder and tubing used to deliver a treatment to a bodily fluid.

FIG. 2 is a partial cross sectional view showing additional detail of the cylinder and tubing of FIG. 1.

### DETAILED DESCRIPTION OF THE INVENTION

In the first stage of treatment, a selected body fluid is removed using a standard catheter and/or lumbar puncture. In the second stage, the body fluid is treated with antibodies against the targeted MS antigen.

The method of the present invention comprises treating at least one component of a patient's body fluid extracorporeally with a designer antibody containing an albumin-moiety to create an albumin-antibody-MS antigen moiety allowing for the efficacious dialysis, filtering or other means of removal of the resultant albumin-antibody-MS antigen compound.

The albumin-antibody will be directed towards facilitating removal of the targeted MS antigen(s): After the removal of the MS antigen(s), the cleansed body fluid will be returned to the patient. The frequency of treatment and the specifically targeted MS antigen(s) to be removed would depend upon the underlying symptomatology and pathology of the patient, and would be determined by the patient's physician.

The article used in performing the method includes two-stages The first stage includes a treatment chamber for addition of an antibody with an attached albumin moiety. which is added to the body fluid. A second stage receives the treated blood and/or CSF and includes a unit for removing the treatment.

The method includes providing a dialysis or other filtering machine with a first stage and a second stage, and sequentially passing the extracorporeal body fluid through the firs and second stages. The body fluid is removed from the patient using standard procedure. The first stage applies a treatment using an antibody which was has attached to it an albumin moiety (or alternatively, a moiety which allows for the efficacious dialysis or removal by other techniques of the antibody-albumin-MS antigen), for the treatment of the body fluid. The second stage substantially removes the treatment. The purified body fluid (body fluid with removed targeted MS antigen(s) is then tested for the efficacy of removal of the MS antigen(s) and returned to the patient.

An alternative methodology of the present: intervention would utilize a designer antibody with an attached macromolecular moiety instead of an albumin moiety. The macromolecular moiety attached to the antibody would have a large size such as, for example, between about 1.000 mm to 0.005 mm in diameter. The large size permits removal of the antibody-macromolecular moiety-targeted antigen complex using physical screen techniques. For example, a series of microscreens can define openings with diameters less than about 50% to more than 99% less than the diameter of the designer antibody-macromolecular moiety. The microscreen opening(s) must have a diameter of at least 25 micrometers in order to allow for the passage and return to circulation of the nonpathologic inducing body fluid constituents.

Alternatively, the target MS antigen(s) may be captured by utilizing antibody microarrays which contain antibodies to targeted MS antigens. The antibody microarrays comprise a plurality of identical monoclonal antibodies attached at high density on glass or plastic slides. Densities can exceed one million microarrays per square centimeter. After sufficient extracorporeal exposure of the targeted MS antigens to the antibody microarrays, the antibody microarrays-targeted MS antigens may be disposed of utilizing standard medical practice.

Another alternative methodology of the pre t intervention comprises removing one or more of the targeted MS antigens from the body fluid by utilizing a designer antibody containing an iron (Fe) moiety. This will then create an Fe-Antibody-Antigen complex. This iron containing complex may then be efficaciously removed utilizing a strong, localized magnetic field.

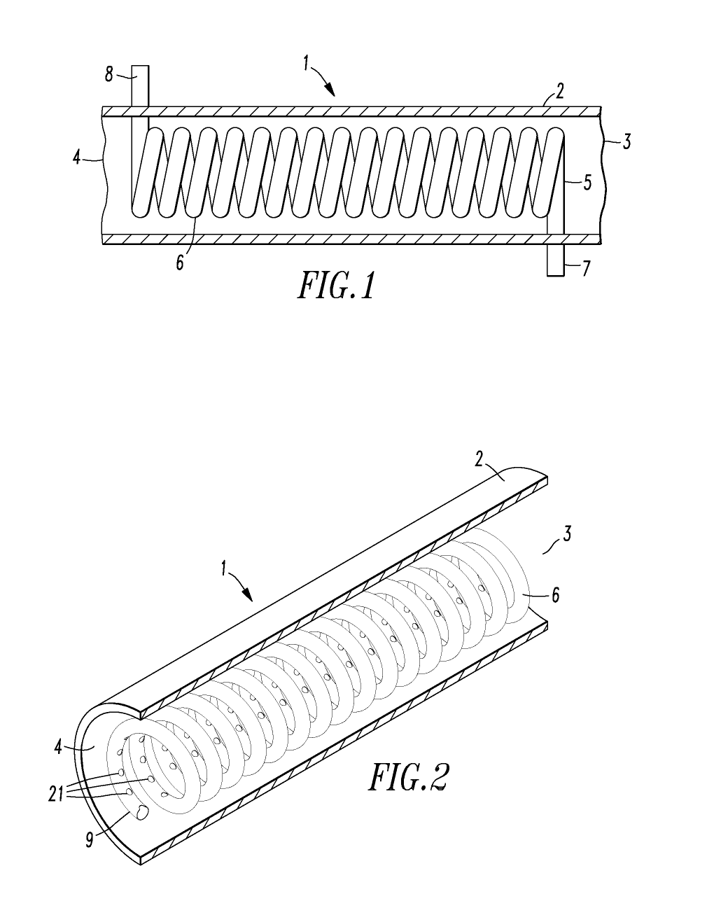

The device of the invention includes a first stage and a second stage. The first stage applies a treatment of an antibody with an attached albumin moiety targeting the MS antigen(s) specifically exacerbating the pathologic condition. The second stage includes substantial removal of the treatment from the extracorporeal body fluid bodily fluid. As shown in FIG. 1, the first stage can include an exterior wall to define a treatment chamber 5. The treatment conveniently can be applied in the treatment chamber 5. Residence times of the body fluid can be altered by changing the dimensions of the treatment chamber, or by using a dialysis vacuum pump. With reference to FIG. 1, body fluid enters the inlet 3, passes through the treatment chamber 5, and exits the outlet 4. In embodiments, the treatment of an antibody with an attached albumin moiety targeting the MS antigen(s) can be applied from a delivery tube 6 located within the treatment chamber 5. An inferior wall 9 defines die delivery tube 6. The delivery tube 6 can include at least one lead 7, 8. The lead 7, 8 can deliver the treatment to the treatment chamber 5. Conveniently, the delivery tubes 6 will have a high contact surface area with the blood and/or CSF. As shown, the delivery tube 6 comprises a helical coil.

With reference to FIG. 2, when the treatment includes the administration of a designer antibody, the delivery tube 6 can be hollow and the interior wall 9 can define a plurality of holes 21. The designer antibodies can be pumped through the delivery tube 6 in order to effect a desired concentration of designer anti bodies in the body fluid. The designer antibodies can perfuse through the holes 21. The delivery tube 6 can include any suitable material including, for example, metal, plastic, ceramic or combinations thereof. The delivery tube 6 can also be rigid or flexible. In one embodiment, the delivery tube 6 is a metal tube perforated with a plurality of holes. Alternatively, the delivery tube 6 can be plastic. The antibody with attached albumin moiety, targeting the MS antigen(s) can be delivered in a concurrent or counter-current mode with reference to the body fluid. In counter-current mode, the body fluid enters the treatment chamber 5 at the inlet 3. The designer antibody can enter through a first lead 8 near the outlet 4 of the treatment chamber 5. The body fluid then passes to the outlet 4 and the designer antibodies pass to the second lead 7 near the inlet 3. The removal module of the second stage substantially removes the designer antibodies-MS antigen molecular compound from the body fluid.

The second stage can include a filter, such as a dialysis machine, which is known to one skilled in the art. The second stage can include a molecular filter including, for example, a molecular adsorbents recirculating system (MARS) that may be compatible and/or synergistic with dialysis equipment. MARS technology can be used to remove small to average sized molecules from the body fluid. Artificial liver filtration presently uses this technique.

The method can include a plurality of steps for removing the targeted MS antigen(s). A first step can include directing a first antibody against the targeted antigen. A second step can include a second antibody. The second antibody can be conjugated with albumin or alternatively another moiety which allows for efficacious dialysis or filtering, of the antibody-MS antigen from the body fluid. The second antibody or antibody-albumen complex combines with the first antibody forming, an antibody-antibody-moiety complex. A third step is then used to remove the complex from the body fluid. This removal is enabled by using dialysis and/or MARS. The purified body fluid is then returned to the patient.

In practice, a portion of the purified body fluid can be tested to ensure a sufficient portion of the targeted MS antigen(s) have been successfully removed from the body fluid. Testing can determine the length of treatment and evaluate the efficacy of the sequential dialysis methodology in removing the targeted MS antigen(s) and suggest the need for further treatment. Body fluid with an unacceptably large concentration of complex remaining can then be retreated and refiltered before returning the body fluid to the patient.

In embodiments, the second stage to remove the antibody-moiety-targeted MS antigen complex from the body fluid can be accomplished by various techniques including, for example, dialysis, filtering based on molecular size, protein binding, solubility, chemical reactivity, and combinations thereof. For example, a filter can include a molecular sieve, such as zeolite, or porous membranes that capture complexes comprising molecules above a certain size. Membranes can comprise polyacrylonitrile, polysulfone, polyanaides, cellulose, cellulose acetate, polyacrylates, polymethylmethacrylates, and combinations thereof. Increasing the low rate or diasylate flow rate can increase the rate of removal of the antibody with attached albumin moiety targeting the MS antigen(s).

Further techniques can include continuous renal replacement therapy (CRRT) which can remove large quantities of filterable molecules from the extracorporeal body fluid. CRRT would be particularly useful for molecular compounds that are not strongly bound to plasma proteins. Categories of cRRT include continuous arteriovenous hemofiltration, continuous venovenous hemofiltration, continuous arteriovenous hemodiafiltration, slow continuous filtration, continuous arteriovenous high-flux hemodialysis, and continuous venovenous high flux hemodialysis. The sieving coefficient (SC) is the ratio of the molecular concentration in the Filtrate to the incoming CSF. A SC close to zero implies that the moiety-antibody-targeted antigen complex will not pass through the filter. A filtration rate of 50 ml per minute is generally satisfactory. Other methods of increasing the removability of the antibody-targeted antigen moiety include the use of temporary acidification of the body fluid extracorporeally using organic acids to compete with protein binding sites.

1. A method for treating an extracorporeal body fluid comprising at least one MS antigen, the method characterized by:
a. combining a first antibody with the MS antigen in the extracorporeal body fluid to produce an antibody-MS antigen moiety; and
b. removing the antibody-MS antigen moiety from the extracorporeal body fluid.

2. The method of claim 1, wherein the MS antigen is selected from a group consisting of integrin, osteopontin, interleukin-23, interleukin-17, interleukin-12, interleukin-intrathecal immunoglobulins IgG/oligoclonal bands, glutamate, matrix metalloproteinases (MMPs), myelin basic protein (MBP), peptidyl arginine deiminase 2 (PAD 2), beta-chemokines monocyte chemoattractant protein-1 (MCP-1), macrophage inflammatory protein (MIP), Regulated on Activation Normal T Cell Expressed (RANTE) and Secreted (CCL5), myelin-associated oligodendrocytic basic protein (MOBP), N-Acetyl-Aspartate, VLA-4 (very late antigen-4). IL15 and LPS-cytokine, Adhesion Proteins, Activated Leukocyte Cell Adhesion Molecule (ALCAM), cluster of differentiation 166 (CD166), chemokine ligand 12 (CXCL12), Endothelin-1, Kallikreins (KLK1, KLK6), Chromogranin A, Myelin Protein TPPP/p25, sFas (soluble form of the Fas molecule), MIF (macrophage migration inhibitory factor), TNF-alpha (tumor necrosis factor-alpha), CCL2 (chemokine ligand 2), T helper cells (Th1 and Th17), Activated T Cells and B Cells, NMO-IgG/Aquaporin-4 Antibodies, Integrin, LINGO-1 (Leucine-rich repeat and Ig domain containing NOGO receptor interacting protein-1), sVCAM-1 (soluble vascular adhesion molecule), A1AC (alpha-1 antichymotrypsin), A2MG (alpha-1 macroglobulin), Fibulin 1, and combinations thereof.

3. The method of claim 1, wherein the MS antigen is selected from a group consisting of integrin, osteopontin, interieukin-23, interleukin-17, glutamate, peptidyl arginine delminase 2 (PAD 2), Regulated on Activation Normal T Cell Expressed (RANTE) and Secreted (CCL5), LINGO-1 (Leucine-rich repeat and Ig domain containing NOGO receptor interacting protein-1), sVCAM-1 (soluble vascular adhesion molecule), A1AC (alpha-1 antiehymotrypsin), A2MG (alpha-1 macroglobulin), Fibulin 1, and combinations thereof.

4. The method of claim 1, wherein the MS antigen is selected from a group consisting of integrin, interleukin-12, interleukin-1, intrathecal immunoglobulins IgG/oligoclonal bands, alutamate, matrix metalloproteinases (MMPs), myelin basic protein (MBP), beta-chemokines monocyte chemoattractant protein-1 (MCP-1), macrophage inflammatory protein (MIP) myelin-associated oligodendrocytic basic protein (MOBP), N-Acetyl-Aspartate, VLA-4 (very late antigen-4), IL15 and LPS-eytokine. Adhesion Proteins, Activated Leukocyte Cell Adhesion Molecule (ALCAM), cluster of differentiation 166 (CD166), chemokine ligand 12 (CXCL12). Endothelin-1, Kallikreins (KLK1, KLK6). Chromogranin A, Myelin Protein TPPP/p25, sFas (soluble form of the Fas molecule), MIF (macrophage migration inhibitory factor), TNF-alpha (tumor necrosis factor-alpha), CCL2 (chemokine ligand 2), T helper cells (Th1 and Th17), Activated T Cells and B Cells, NMO-IgG/Aquaporin-4 Antibodies, and combinations thereof.

5. The method of claim 1, characterized by removing the antibody-MS antigen moiety includes irradiation, magnetism, mechanical filtering, chemical filtering, and combinations thereof.

6. The method of claim 1, further characterized by conjugating the antibody-MS antigen with albumin thereby forming an albumin-antibody-MS antigen compound.

7. The method of claim 1 further characterized by testing the extracorporeal body fluid for efficacy of removing the antibody-MS antigen moiety.

8. The method of claim 1 Further characterized by removing a body fluid from a patient to produce the extracorporeal body fluid and returning the extracorporeal body fluid to the patient after treating the extracorporeal body fluid.

9. The method of claim 1, characterized by combining the first antibody with the MS antigen in a first stage, passing the extracorporeal body fluid to a second stage, and removing the antibody-MS antigen moiety from the body fluid in the second stage.

10. The method of claim 9, characterized by providing a filtering machine comprising the first stage and the second stage, and sequentially passing the extracorporeal body fluid through the first and second stages.

11. The method of claim 9, characterized by conjugating the antibody-MS antigen with albumin in the first stage, thereby forming an albumin-antibody-MS antigen compound.

12. The method of claim 1, characterized by conjugating the antibody-MS antigen with a designer antibody comprising an attached macromolecular moiety, thereby forming an antibody-macromolecular moiety-targeted antigen complex having a diameter.

13. The method of claim 12, characterized by the diameter of the antibody-macromolecular moiety-targeted antigen complex being from about 0.005 mm to 1.000 mm.

14. The method of claim 12, characterized by removing the antibody-macromolecular moiety-targeted antigen complex by filtering through at least one screen filter defining a plurality of openings having opening diameters less than the diameter of the antibody-macromolecular moiety-targeted antigen complex.

15. The method of claim 1, characterized by the first antibody being fixed to an antibody microarray, whereby removing the antibody-MS antigen moiety from the extracorporeal body fluid comprises fixing the antibody-MS antigen moiety to the microarray.

16. The method of claim 1, characterized by combining the antibody-MS antigen moiety with at toast one antibody containing iron, thereby forming an Fe-Antibody-Antigen complex, and removing the Fe-Antibody-Antigen complex using a strong, localized magnetic field.

17. The method of claim 1, characterized by removing the antibody-MS antigen moiety using Kanzius radiofrequency (RF) therapy and removing residue of the Kanzius radiofrequency (RF) therapy from the extracorporeal body fluid.

18. The method of claim 1, characterized by removing the antibody-MS antigen moiety using a molecular filter.

19. The method of claim 1, characterized by removing the antibody-MS antigen moiety using a molecular sieve comprising a material selected from a group consisting of zeolite, polyacrylonitrile, polysulfone, polyamide, cellulose, cellulose acetate, polyacrylate, polymethylmethacrylate, and combinations thereof

20. The method of claim 1, further characterized by retreating the extracorporeal body fluid if an unacceptably large concentration of antibody-MS antigen moiety remains in the extracorporeal body fluid.
